(19) United States
(12) Patent Application Publication
(10) Pub. No.: US 2025/0040811 A1
(43) Pub. Date: Feb. 6, 2025

(54) Method and apparatus for smart respiratory monitoring by electrocardiogram, breath acoustics and thoracic acceleration

(71) Applicant: CORE SAFE MEDICAL SL, Barcelona (ES)

(72) Inventor: HECTOR LITVAN, Barcelona (ES)

(21) Appl. No.: 18/298,399

(22) Filed: Apr. 11, 2023

Related U.S. Application Data
(63) Continuation of application No. PCT/IB21/59526, filed on Oct. 15, 2021.

(30) Foreign Application Priority Data
Oct. 20, 2020 (DK) PA 2020 01182

(51) Int. Cl. A61B 5/0205; A61B 5/00; A61B 5/024; A61B 5/08; A61B 5/091; A61B 5/113; A61B 5/308; A61B 5/318; A61B 7/00

(52) U.S. Cl. CPC A61B 5/0205; A61B 5/1135; A61B 5/308; A61B 5/318; A61B 5/6833; A61B 5/7257; A61B 5/7264; A61B 7/003; A61B 5/02405; A61B 5/0816; A61B 5/091; A61B 2562/0204; A61B 2562/0219

(57) ABSTRACT

Respiration is fundamental for life. The continuous monitoring of respiratory rate and its pattern is crucial for detecting the onset of respiratory failure; however, most hospital wards fail to monitor this vital sign. Respiratory distress results from failure of oxygenation (insufficient inhalation of oxygen), or failure of ventilation (insufficient removal of carbon dioxide). The present invention discloses methods and apparatus for monitoring of the respiratory rate by extraction of information from the thoracic acceleration, the electrocardiogram, and the breath sounds. The present invention also discloses a method quantifying the deterioration of the respiration by defining the Smart Respiratory Index by combining at least three parameters extracted from physiological measurements such as the electrocardiogram, breath sounds and thoracic acceleration. The methods are implemented in a small wireless patch attached to the upper part of the thorax of the patient and communicate by bluetooth to an external proprietary software and monitor.

### FIELD OF THE INVENTION

The present invention generally relates to a device and method for determining the quality and effort of respiration and the respiratory rate (RR) of a subject. More particularly, the present invention pertains to a wireless device and method for obtaining an index presenting the probability of deterioration of the respiration.

### BACKGROUND

Respiration is fundamental for life. The lungs are responsible for respiration, the process of supplying oxygen to the body and removing carbon dioxide from the body. Respiratory distress results from failure of oxygenation (insufficient inhalation of oxygen), or failure of ventilation (insufficient removal of carbon dioxide).

These forms of failure can be characterized by irregular breathing rate and/or abnormal breathing patterns. The continuous monitoring of RR and pattern is crucial for detecting the onset of respiratory failure; however, most hospital wards fail to correctly monitor this vital sign. When RR is measured, it is done infrequently. Patients in the general ward are typically checked only once every six to eight hours and often with significant human error, due to the fact that normally the nurse watches the patient breathe for 15 s and then multiply the result by 4 in order to get the RR per min. The World Health Organization promotes to count the number of breaths in one minute. If the RR is higher or lower than the accepted range of 12-16 breaths/min it suggests respiratory issues.

Current devices for measuring RR are very expensive and are connected to the patient with cables, hence they are only used in specialized units such as the Intensive Care Units (ICU).

Twenty-one percent of the patients with RR between 25 and 29/min die in the hospital (Respiratory rate: the neglected vital sign. Michelle A Cretikos, Rinaldo Bellomo, Ken Hillman, Jack Chen, Simon Finfer and Arthas Flabouris. MIA 2008; 188:657-659) The best way to alert the clinical state of the patient and reduce the complications are RR and tidal volume (TV) continuous monitoring. The Corona virus pandemic (Covid-19) has made it clear that respiratory monitoring is necessary outside the hospital setting in nursing homes and even in a home care setting.

The US patent application, “Acoustic sensor and ventilation monitoring system”, US 2020/0054277 A1, by Joseph et al., discloses a method of monitoring respiration with an acoustic measurement device. After an extensive description of the respiratory physiopathology the authors present a device integrated by two elements. One of them is attached to the patient while the second element is attached to the first element. The first element consists of a sound transducer, an accelerometer and a transmitter. The second element consists of a rechargeable battery. In the device several sensors can be integrated to measure temperature, heart rate and oxygen saturation. It could also be connected to a smart watch. With the information from the different sensors they propose a risk factor index to determine the function of the respiration of a mammalian due to physiopathological alterations and drugs or alcohol abuse. They describe a quadratic equation to calculate their respiratory risk factor index. The main differences with our present patent application are on one hand that Joseph et al. use the accelerometer only to assess the body movement while we use the accelerometer for estimation of the respiratory frequency and on the other hand the formulas for respiratory risk index and smart respiratory index are significantly different. Additionally, in the patent application by Joseph et al. the respiratory rate is estimated by the breath acoustics while in the present patent application the respiratory rate is estimated by the thoracic acceleration measured by the accelerometer and the first derivative of the breath sound envelope.

Both the patent application by Joseph and the present patent application use sensors as microphones and accelerometers to assess the sound of breathing and the movement. However, the parameters analyzed in the two works and the derived formula to calculate the risk factor index by Joseph et al and the SRI in the present disclosure are completely different. Joseph et al use the accelerometer to assess the body movement of the patient while in this disclosure the accelerometer is used to calculate the respiratory rate.

Joseph et al use the breath sounds to calculate the respiratory rate and the TV and in this disclosure is used the envelope to assess the variability of the TV.

Finally, for Joseph et al the body movements and the speech add up the highest score while the bradypnea and the lack of movements have the lowest possible score.

The US application, “Portable device with multiple integrated sensors for vital signs scanning”, US2015/0313484 A1, discloses a portable device with multiple integrated sensors. The present patient application is different as it does not use neither thermometer nor photoplethy smogram (PPG) and the present application defines an index of the quality of respiration.

The US application, “Mobile frontend system for comprehensive cardiac diagnosis.” US2015/0065814 A1 is significantly different from the present patent application because the purpose of US2015/0065814 is comprehensive diagnosis of heart issues.

The US patent application, “Mesh network personal emergency appliance” US 2008/0001735 A1 discloses a system that includes one or more wireless nodes forming a wireless mesh network, the present application is different as it does not form a wireless mesh network.

The US patent application, “Monitoring, predicting and treating clinical episodes”, JS2008/0275349 A1”, discloses a device for sensing a physiological parameter of a subject and to sense large body movements, this is significantly different from the present patent application which does not disclose sensing of large body movements. The US application, “Physiological acoustic monitoring system, U.S. Pat. No. 8,821,415B2 United States, is 25 disclosing a method to use acoustic signals to assess the respiratory rate, however they only use one or two acoustic sensors (microphones), hence the present application is totally different as electrocardiogram (ECG) and an accelerometer are used as well.

The US patent METHODS AND SYSTEMS FOR MONITORING RESPIRATION U.S. Pat. No. 6,918,878B2 discloses a method for determining respiration rate in a patient including various parts. The respiration rate can be determined by measuring the heart's S2 split. The S2 split can be identified by observing the timing of the heart sounds. Other respiration related information, such as respiration phase and the occurrence of apnea, can be identified as well A respiration monitor of this type may be useful for monitoring sub-acute patients, and outpatients. A sensor for the respiration monitor and an electrode for an ECG monitor may be combined into a single probe. The present patent application does not include the S2 split and is hence different from U.S. Pat. No. 6,918,878 patent application. The US patent, “System and method for monitoring respiratory rate measurements”, US 2018/0214090 A1, disclose systems and methods for using multiple physiological parameter inputs to determine multiparameter confidence in respiratory rate measurements. This disclosure is different from the present patent application as breath acoustic and ECG R-R interval variation (Rpeaks) is combined in the present application.

### SUMMARY OF THE INVENTION

In a preferred embodiment a patch (1, 13, 27, 42) containing sensors for at least electrocardiogram (ECG), Respiratory Rate (RR) measured by an accelerometer, RR measured by a microphone is attached to the thorax of the patient. The ECG (2, 14, 29) is further processed with a heart rate (HR) extraction algorithm, for example but not necessarily a Fast Fourier Transform (FFT) (6, 19, 34) to extract the HR of the ECG and the Rpeaks (7, 20, 35) are determined by FFT as well (spectral analysis). The breath sounds (breathing acoustics) from the microphone (3, 15, 30) are subjected to an envelope wave form respiration extraction formula (8, 21, 36) to obtain the respiratory rate, termed RespR. The formula (9, 22, 37) is used to calculate the tidal volume variability (TVv). The acceleration signal of the thorax (4, 16, 31) is analyzed by a Hilbert Transform model which estimates the respiration, termed RespRacc (10, 23, 38). The relationship among the three parameters, Rpeaks, RespR and RespRacc might change as the respiration is deteriorating, therefore the Cross Mutual information (5, 18, 32) is calculated and generating the variable CMIbreath. The parameters extracted from the measurements are fed into a classifier, which could, but not necessarily, be an Adaptive Neuro Fuzzy Inference System (ANFIS) (11, 25, 40). The output of the classifier is termed the Smart Respiratory Index (SRI) (12, 26, 41).

### Estimation of the RespR by an Envelope Formula.

The acoustic signal recorded from the microphone is entered into a spline or other curve function which allows to assess the envelope of the amplitude, Aenvelope, as shown in FIG. 7. By counting the peaks in the Aenvelope curve the RespR can be calculated, see also the example in FIG. 6.

### Estimation of Tidal Volume Variability (TVv)

Previously, the relationship between respiratory airflow, F, and the energy of breath (tracheal) sound, E, was best fitted by a power law of the form A=kF” where k and a are constants, where different values have been suggested for the exponent by different research groups. This sound's amplitude-airflow relationship has been exploited for breathing monitoring, particularly for qualitative and quantitative assessment of respiratory airflow and for continuous respiratory rate estimation.

However, we found that the estimation can be improved by adding the derivative of the envelope of the breath sound to the equation, hence the following equation for flow is defined:

$$F = {k}_{1} max ( dAenvelope / dt ) + {k}_{2} {A}^{beta}$$

Consequently, the volume can be estimated as the integral of the airflow F over the time of inspiration,

TV=∫_{insp start}^{insp end}Fdt

The tidal volume variability is here defined as the changes over time, for example if the tidal volume increases from 10 to 12 then the tidal volume variability is 20%.

### Determination of the RespRacc by Applying the Hilbert Transform to the Acceleration Signal.

A new approach using the Hilbert vibration decomposition (I-IVD) for extracting the respiration from the acceleration signal is proposed. It is suggested that the largest energy component of the acceleration signal acquired is proportional to the respiratory signal.

### Determination of the Smart Respiratory Index, (SRI)

In one embodiment the SRI is a function, linear or quadratic, of the RespR, HR, RRv and TVv. The formula could be,

$$SRI = k 1 * RespR + k 2 * HR + k 3 * RRV + k 4 * TVv + k 5 * RespR * HR * RRV * TVv .$$

In the quadratic equation more quadratic factors could be included.

The constants, k1 to k4, should be in the following range:
0.30<k1<0.7, 0.2<k2<0.4,
0.05<k3<0.2,
0.01<k4<0.1.

### Combination of Parameters by a Classifier for Defining the SRI.

In a second embodiment the device uses a classifier such as, but not necessarily, an ANFIS model to combine the parameters, for the definition of the SRI, as shown in FIG. 1. The parameters extracted from the at least 3 sensors (ECG, breath sound, thoracic acceleration) are used as input to an Adaptive Neuro Fuzzy Inference System (ANFIS). In a third embodiment the device uses ANFIS models to combine the parameters, for the definition of the SRI, as shown in FIG. 2. The parameters extracted from the at least 4 sensors (ECG, breath sound, thoracic acceleration, pulse oximeter) are used as input to an Adaptive Neuro Fuzzy Inference System (ANFIS).

In a fourth embodiment the device uses ANFIS models to combine the parameters, for the definition of the SRI, as shown in FIG. 3. The parameters extracted from the at least 4 sensors (ECG, breath sound, thoracic acceleration, pulse oximeter) and the demographic (age, sex, height, weight) and clinical data (chronic pulmonary obstructive disease, asthma, sympathetic disorders, atrial fibrillation, beta-blockers, pacemaker) of the patient are used as input to an Adaptive Neuro Fuzzy Inference System (ANFIS).

### Overview of ANFIS

ANFIS is a hybrid between a fuzzy logic system and a neural network, it does not assume any mathematical function governing the relationship between input and output. ANFIS applies a data driven approach where the training data decides the behaviour of the system.

The five layers of ANFIS have the following functions
Each unit in Layer 1 stores three parameters to define a bell-shaped membership function. Each unit is connected to exactly one input unit and computes the membership degree of the input value obtained.
Each rule is represented by one unit in Layer 2. Each unit is connected to those units in the previous layer, which are from the antecedent of the rule. The inputs into a unit are degrees of membership, which are multiplied to determine the degree of fulfilment for the rule represented.
In Layer 3, for each rule there is a unit that computes its relative degree of fulfilment by means of a normalization equation. Each unit is connected to all the rule units in Layer 2.
The units of Layer 4 are connected to all input units and to exactly one unit in Layer 3. Each unit computes the output of a rule.
An output unit in Layer 5 computes the final output by summing all the outputs from Layer 4.

Standard learning procedures from neural network theory are applied in ANFIS. Back-propagation is used to learn the antecedent parameters, i.e the membership functions, and least squares estimation is used to determine the coefficients of the linear combinations in the rules' consequents. A step in the learning procedure has two passes. In the first pass, the forward pass, the input patterns are propagated, and the optimal consequent parameters are estimated by an iterative least mean squares procedure, while the antecedent parameters are fixed for the current cycle through the training set. In the second pass, the backward pass, the patterns are propagated again, and in this pass back-propagation is used to modify the antecedent parameters, while the consequent parameters remain fixed. This procedure is then iterated through the desired number of epochs if the antecedent parameters initially are chosen appropriately, based on expert knowledge, one epoch is often sufficient as the LMS algorithm determines the optimal consequent parameters in one pass and if the antecedents do not change significantly by use of the gradient descent method, neither will the LMS calculation of the consequents lead to another result. For example in a 2-input, 2-rule system, rule 1 is defined by

$$if x is A and y is B then {f}_{1} = {p}_{1} x + {q}_{1} y + {r}_{1}$$

where p, q and r are linear, termed consequent parameters or only consequents. Most common is f of first order as higher order Sugeno fuzzy models introduce great complexity with little obvious merit.

### Number of Classes.

The inputs to the ANFIS system are fuzzified into a number of predetermined classes. The number of classes should be larger or equal to two. The number of classes can be determined by different methods. In traditional fuzzy logic the classes are defined by an expert. The method can only be applied if it is evident to the expert where the landmarks between two classes can be placed. ANFIS optimizes the position of the landmarks, however the gradient descent method will reach its minimum faster if the initial value of the parameters defining the classes is close to the optimal values. By default, ANFIS initial landmarks are chosen by dividing the interval from minimum to maximum of all data into n equidistant intervals, where n is the number of classes. The number of classes could also be chosen by plotting the data in a histogram and visually deciding for an adequate number of classes, by ranking as done by fuzzy inductive reasoning (FIR), through various clustering methods or Markov models. The ANFIS default was chosen for this invention and it showed that more than 3 classes resulted in instabilities during the validation phase, hence either 2 or 3 classes were used.

### Number of Inputs.

Both the number of classes and number of inputs add to the complexity of the model i.e. the number of parameters. For example, a system with 4 inputs, each fuzzified into 3 classes consists of 36 antecedent (non-linear) and 405 consequent (linear) parameters, calculated by the following two formulas:

$$antecedents = number of classes \times number of inputs \times 3$$

$$consequents = number of {classes}^{number of inputs} \times ( number of inputs + 1 )$$

The number of input-output pairs should in general be much larger, (at least a factor 10) than the number of parameters in order to obtain a meaningful solution of the parameters.

### Stability Criteria.

Unfortunately there exists no definition of stability criteria for neuro-fuzzy systems. The most useful tool for ensuring stability is the experience obtained by working with a certain neuro-fuzzy system such as ANFIS in the context of a particular data set, and testing with extreme data for example obtained by simulation.

### Number of Epochs.

ANFIS uses a Root Mean Square Error (RMSE) to validate the training result and from a set of validation data the RMSE validation error can be calculated after each training epoch. One epoch is defined as one update of both the antecedent and the consequent parameters. An increased number of epochs will in general decrease the training error.

### Legends to Figures

FIG. 1. The extracted parameters are fed into an ANFIS which is a hybrid between a neural network and a fuzzy logic system. The inputs are at least 3 parameters among the following: HR (6), RRv (7), RespR (8), TVv (9), RespRacc (10), Cross Mutual Information among the HR, RRv and the RespRacc (CMlbreath) (5). The output of the ANFIS model (11) is the Smart Respiratory Index (SRI) (12), which is a unitless number from 0 to 100, where a decreasing value corresponds to a deterioration of the patient's respiratory function.

FIG. 2. The extracted parameters are fed into an ANFIS which is a hybrid between a neural network and a fuzzy logic system. The inputs are at least 3 parameters among the following: HR (19), RRv (20), RespR (21), TVv (22), RespRacc (23), pulseoximetry (17), Cross Mutual Information among the HR, RRv and the RespRacc (CMlbreath) (18). The output of the ANFIS (25) model is the Smart Respiratory Index (SRI) (26), which is a unitless number from 0 to 100, where a decreasing value corresponds to a deterioration of the patient's respiratory function.

FIG. 3. The extracted parameters are fed into an ANFIS which is a hybrid between a neural network and a fuzzy logic system. The inputs are at least 3 parameters among the following: HR (34), RRv (35), RespR (36), TVv (37), RespRacc (38), pulseoximetry (39), Cross Mutual Information among the HR, RRv and the RespRacc (CMIbreath) (32), demographic data such as sex, age and body mass index (BMI). The output of the ANFIS model (40) is the Smart Respiratory Index (SRI) (41), which is a unitless number from 0 to 100, where a decreasing value corresponds to a deterioration of the patient's respiratory function FIG. 4. This figure represents how the respiratory patch (42) is attached to the thorax of a human being and the position of the patch.

FIG. 5. The patch consists of an amplifier (43) for ECG (51), an accelerometer (52), a microphone (53), a radio transmitter module for example a bluetooth low energy module (46), a battery (45) and four electrodes (47-50) which simultaneously serve for attaching the patch to the patient. FIG. 6. This figure represents the digital processing of the acquired signals such as ECG (54), breath sounds (55) and thoracic movement (56). It also shows the obtained parameters from each signal, from the ECG (54) we obtain the HR. RRv, from the microphone (55) the RespR, TVv, and from the accelerometer (56) we obtain the RespRacc

FIG. 7. This figure shows a schematic representation of the cyclic breath sound of inspiration and expiration, where the amplitude envelope has been plotted as well.

FIG. 8. The table represents the relationship between the clinical state and the Smart Respiratory Index (SRI). The SRI is a gradual scale where 100 corresponds to normal respiratory function, while decreasing values reflect a deterioration of the breathing function and where 0 is presented when respiratory arrest occurs.

FIG. 9. This figure represents one of the graphical user interfaces (GUI) of the display, where the SRI is the most important parameter and hence the largest. The GUI also shows the RR and the HR.

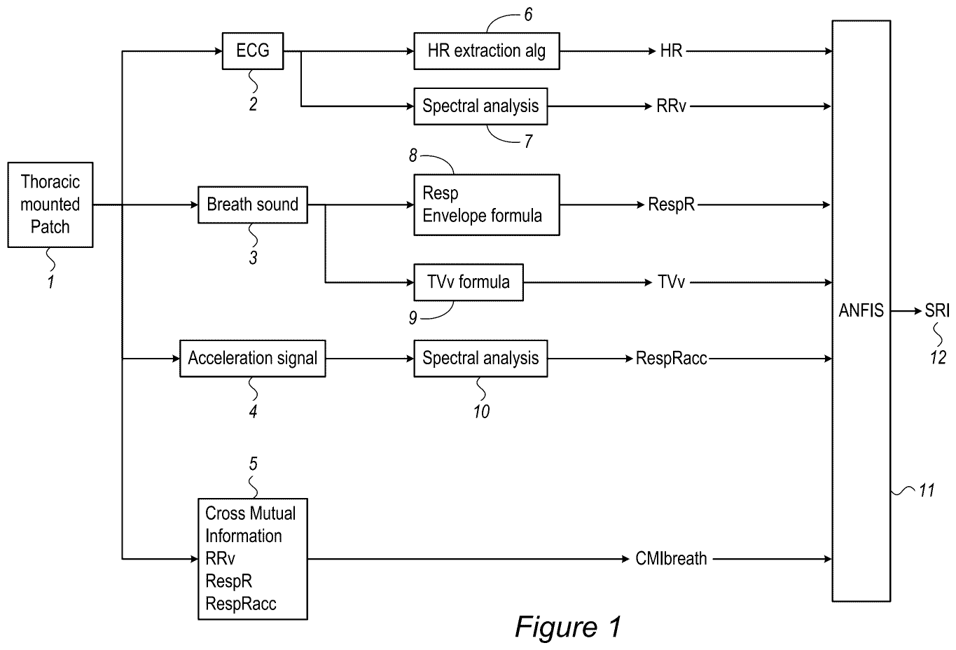

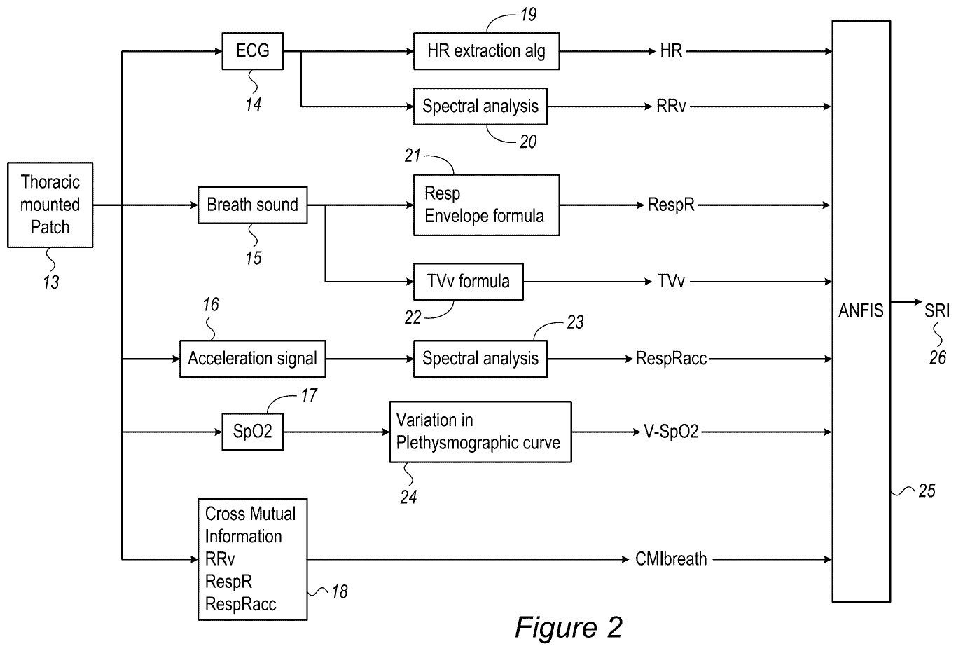

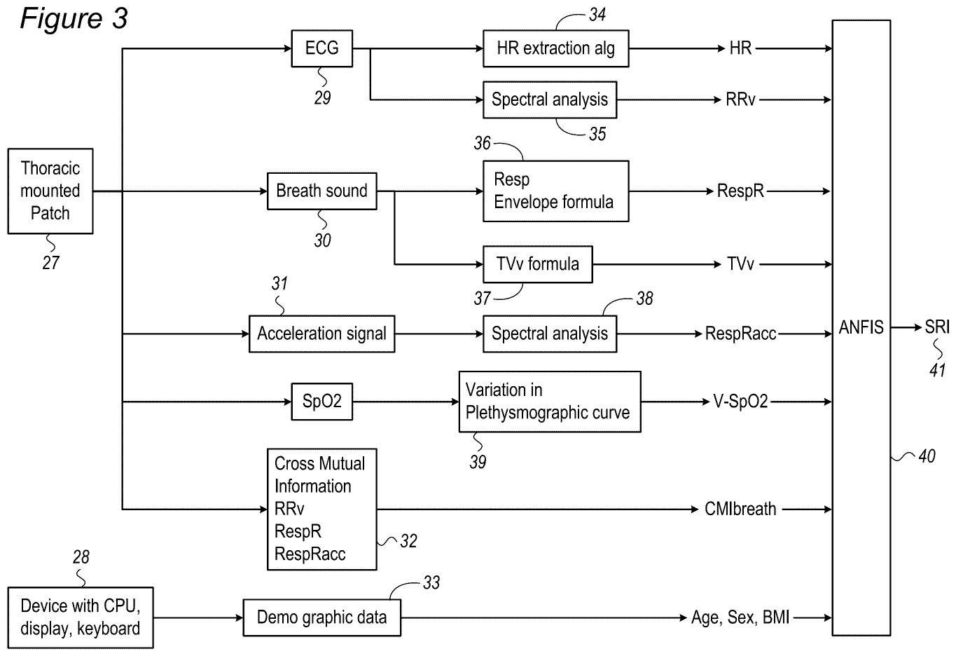

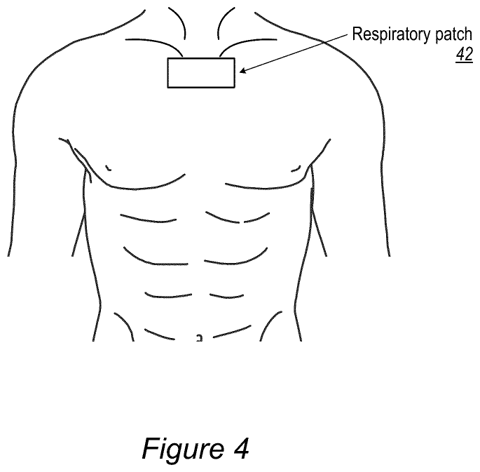

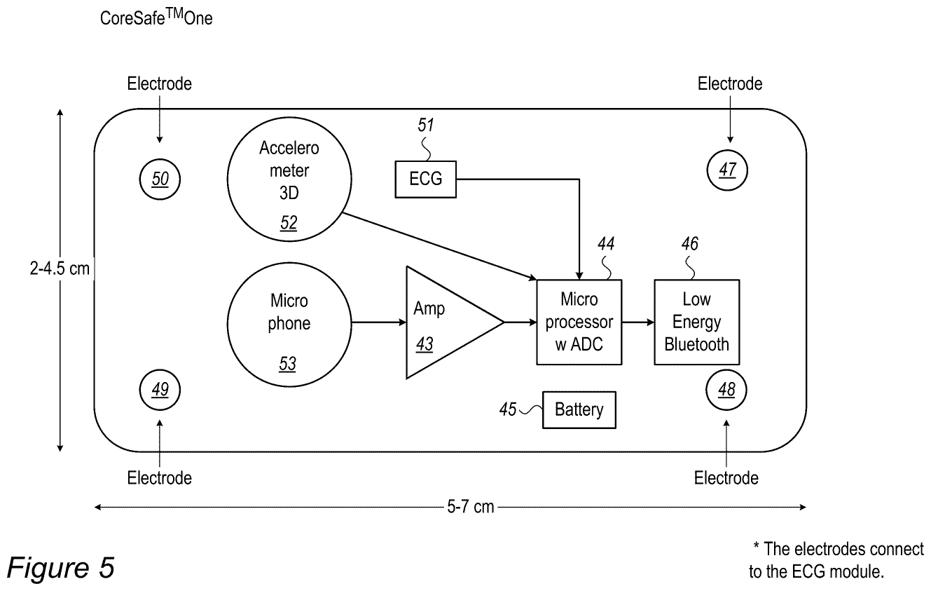

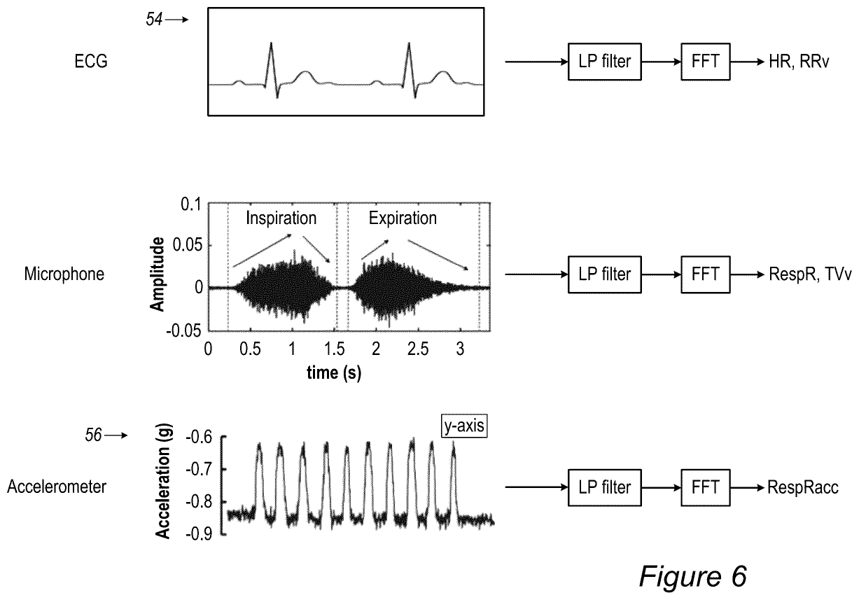

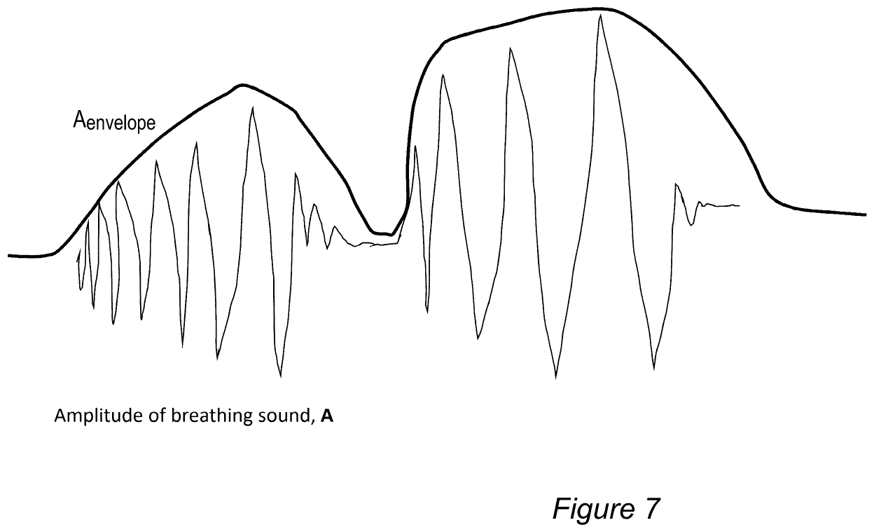

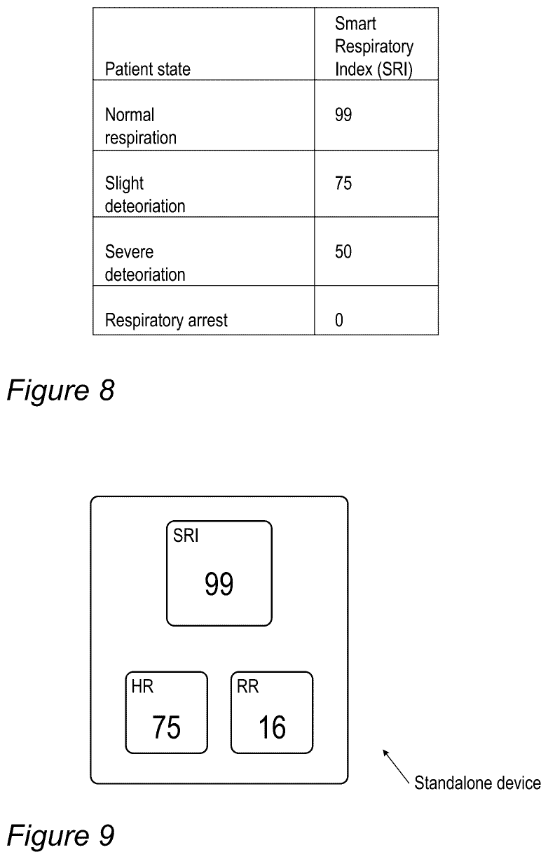

1. A method for determining the quality of respiration of the patient by combination of parameters extracted from an electrocardiogram (2, 14, 29), a thoracic movement and acceleration (4, 16, 31) and a breathing acoustic (3,15,30), characterized by the following steps:
a) measuring the electrocardiogram (2, 14, 29) using a sensor;
b) measuring the thorax movements and acceleration (4, 16, 31) using a sensor;
c) measuring the breathing acoustics (3, 15, 30) using a sensor;
d) calculating a heart rate variability (7, 20, 35) from the electrocardiogram (2, 14, 29) using a Fast Fourier Transform or a Choi-Williams distribution;
e) calculating a tidal volume variability (9, 22, 37) based on the amplitude and a first derivative of an amplitude envelope of the breathing acoustics (8, 21, 36);
f) calculating a respiratory rate from the thorax movements and acceleration (10, 23, 38):
g) calculating a cross mutual information (5, 18, 32) between the heart rate variability of the electrocardiogram (7, 20, 35), breathing acoustics (3, 15, 30), thoracic movements (4, 16, 31), and the tidal volume variability (9, 22, 37) as an input to a smart respiratory index (12, 26, 41);
h) combining at least 3 extracted parameters from the electrocardiogram (2, 14, 29), the breathing acoustics (3, 15, 30), the thoracic movements (4, 16, 31) and the cross mutual information (5, 18, 32) of the three, using an Adaptive Neuro Fuzzy Inference System (11, 25, 40) or any other classifier into an index of level of quality of respiration (12, 26, 41).

2. The method according to claim 1 wherein step a is characterized by measuring by a patch consisting of 2 or more electrodes to determine the electrocardiogram (2, 14, 29) positioned either on the upper or lower thorax of the subject.

3. The method according to claim 1 wherein step b is characterized by recording of the thoracic acceleration by an accelerometer (4, 16, 31) integrated in the patch in order to calculate the respiratory rate based on acceleration (10, 23, 38).

4. The method according to claim 1 wherein step c is characterized by recording of the breathing acoustics (3, 15, 30), inspiration and expiration, by a microphone integrated in the patch (53), in order to calculate the respiratory rate (8, 21, 36).

5. The method according to claim 1 wherein step e is characterized by estimating the flow by the formula,
$$F = {k}_{1} max ( dAenvelope / dt ) + {k}_{2} {A}^{beta}$$
where F is the flow and A the amplitude of the breathing acoustics (3, 15, 30), hence the tidal volume variability (9, 22, 37) is the integral of the flow over time,
TV=∫_{insp start}^{insp end}Fdt.

6. The method according to claim 1 wherein step f is characterized by calculation of the respiratory rate applying a Hilbert transform to the acceleration signal for extracting the respiration from the acceleration (10, 23, 38) where the largest energy component of the acceleration signal acquired is proportional to the respiratory rate.

7. The method according to claim 1 wherein step g is characterized by calculating the cross mutual information (5, 18, 32) among the heart rate variability (7, 20, 35) extracted from the electrocardiogram (2, 14, 29), the features extracted from the breathing acoustics (3, 15, 30) and the features extracted from the acceleration (4, 16, 31).

8. The method according to claim 1 wherein step h is characterized by the extraction of the heart rate variability (7, 20, 35) from the electrocardiogram (2, 14, 29) and the calculation of the tidal volume variability (9, 22, 37) from the breathing acoustics (3, 15, 30), the cross mutual information (5, 18, 32) are used as input to an Adaptive Neuro Fuzzy Inference System (11, 25, 40) or another classifier whose output is an index of the quality of respiration (12, 26, 41).

9. The method according to claim 1 wherein step h is characterized by a formula of the index of quality of respiration (12, 26, 41) as a function, linear or quadratic, of the 14 Respiratory Rate (8, 21, 36), Heart Rate, (6, 19, 34) Heart Rate Variability (7, 20, 35) and Tidal Volume variability (9, 22, 37),
$$SRI = k 1 * RespR + k 2 * HR + K 3 * RRV + k 4 * TVv + k 5 * RespR * HR * RRV * TVv ,$$
where the constants, k1 to k4, should be in the following range:
0.30<k1<0.7,
0.2<k2<0.4,
0.05<k3<0.2,
0.01<k4<0.1.

10. The method according to claims 1 to 10 are integrated in a wireless patch (1, 13, 27, 42) containing interconnected electrocardiogram sensor (51) and amplifier (43), microphone (53), accelerometer (52), battery (55) and a radio transmitter such as a bluetooth low energy module (46) transmitting the data to an external viewer device (57).

11. An apparatus for determining the quality of respiration of a patient, wherein the apparatus is configured for combining parameters extracted from an electrocardiogram (2, 14, 29), a thoracic movement and acceleration (4, 16, 31) and a breathing acoustic (3, 15, 30), characterized by the following steps:
a) measuring the electrocardiogram (2, 14, 29) using a sensor,
b) measuring the thorax movements and acceleration (4, 16, 31) using a sensor;
c) measuring the breathing acoustics (3, 15, 30) using a sensor;
d) calculating a heart rate variability (7, 20, 35) from the electrocardiogram (2, 14, 29) using a Fast Fourier Transform or a Choi-Williams distribution,
e) calculating a tidal volume variability (9, 22, 37) based on the amplitude and a first derivative of an amplitude envelope of the breathing acoustics (8, 21, 36);
f) calculating a respiratory rate from the thorax movements and acceleration (10, 23, 38);
g) calculating a cross mutual information (5, 18, 32) between the heart rate variability of the electrocardiogram (7, 20, 35), breathing acoustics (8, 21, 36), thoracic movements (4, 16, 31), and the tidal volume variability (9, 22, 37) as an input to a smart respiratory index (12, 26, 41);
h) combining at least 3 extracted parameters from the electrocardiogram (2, 14, 29), the breathing acoustics (3, 15, 30), the thoracic movements (4, 16, 31) and the cross mutual information (5, 18, 32) of the three, using an Adaptive Neuro Fuzzy Inference System (11, 25, 40) or any other classifier into an index of level of quality of respiration (12, 26, 41).

12. The apparatus according to claim 11 wherein step a is characterized by a patch (1, 13, 27, 42) consisting of 2 or more electrodes (47, 48, 49, 50) to determine the electrocardiogram (2, 14, 29) positioned either on the upper or lower thorax of the subject.

13. The apparatus according to claim 11 wherein step b is characterized by configuring the apparatus for recording of the thoracic acceleration by an accelerometer (4, 16, 31) integrated in the patch (42) in order to calculate the respiratory rate based on acceleration (10, 23, 38).

14. The apparatus according to claim 1 wherein step c is characterized by configuring the apparatus for recording of the breathing acoustics (3, 15,30), inspiration and expiration, by a microphone (53) integrated in the patch (1, 13, 27, 42), in order to calculate the respiratory rate (8, 21, 36).

15. The apparatus according to claim 1 wherein step e is characterized by configuring the apparatus for estimating the flow by the formula,
$$F = {k}_{1} max ( dAenvelope / dt ) + {k}_{2} {A}^{beta}$$
where F is the flow and A the amplitude of the breathing acoustics, hence the tidal volume variability is the integral of the flow over time,
TV=∫_{insp start}^{insp end}Fdt.

16. The apparatus according to claim 11 wherein step f is characterized by configuring the apparatus for calculation of the respiratory rate applying a Hilbert transform to the acceleration signal (4, 16, 31) for extracting the respiration from the acceleration (10, 23, 38) where the largest energy component of the acceleration signal acquired is proportional to the respiratory rate.

17. The apparatus according to claim 11 wherein step g is characterized by configuring the apparatus for calculating the cross mutual information (5, 18, 32) among the heart rate variability (7, 20, 35) extracted from the electrocardiogram (2, 14, 29), the features extracted from the breathing acoustics (3, 15, 30) and the features extracted from the acceleration (4, 16, 31).

18. The apparatus according to claim 11 wherein step h is characterized by configuring the apparatus for the extraction of the heart rate variability from the electrocardiogram (7, 20, 35) and the calculation of the tidal volume variability from the breathing acoustics (9, 22, 37), the cross mutual information (5, 18, 32) are used as input to an Adaptive Neuro Fuzzy Inference System (11, 25, 40) or another classifier whose output is an index of the quality of respiration (12, 26, 41).

19. The apparatus according to claim 1 wherein step h is characterized by a formula of the index of level of quality of respiration (12, 26, 41) as a function, linear or quadratic, of the Respiratory Rate (8, 21, 36), Heart Rate (6, 19, 34), Heart Rate variability (7, 20, 35) and Tidal Volume variability (9, 22, 37),
$$SRI = k 1 * RespR + k 2 * HR + K 3 * RRV + k 4 * TVv + k 5 * RespR * HR * RRV * TVv ,$$
where the constants, k1 to k4, should be in the following range:
0.30<k1<0.7,
0.2<k2<04,
0.05<k3<0.2,
0.01<k4<0.1.

20. The apparatus according to claims 11 to 19 are integrated in a wireless patch (1, 13, 27, 42) containing interconnected electrocardiogram sensor (51) and amplifier (43), microphone (53), accelerometer (52), battery (45) and a radio transmitter such 17 as a bluetooth low energy module (46) transmitting the data to an external viewer device (57).
